(19) United States
(12) Patent Application Publication
(10) Pub. No.: US 2023/0314211 A1
(43) Pub. Date: Oct. 5, 2023

(54) RADIOGRAPHING APPARATUS

(71) Applicant: CANON KABUSHIKI KAISHA, Tokyo (JP)

(72) Inventors: ATSUSHI TAKEUCHI, Kanagawa (JP); SATORU SAWADA, Kanagawa (JP)

(21) Appl. No.: 18/194,078

(22) Filed: Mar. 31, 2023

(30) Foreign Application Priority Data
Apr. 5, 2022 (JP) 2022-062973

(51) Int. Cl. G01J 1/42; A61B 6/00

(52) U.S. Cl. CPC G01J 1/429; A61B 6/4233

(57) ABSTRACT

A radiographing apparatus includes a radiation detector configured to detect an incident radioactive ray and convert the detected radioactive ray into an electrical signal related to a radiation image, and a housing configured to contain the radiation detector, wherein an improvement to the radiographing apparatus is that an ultraviolet light transmitting member having transmittivity for ultraviolet light is provided at least in a part of an outer housing surface of the housing.

### BACKGROUND OF THE INVENTION

### Field of the Disclosure

The present disclosure relates to a radiographing apparatus that detects incident radioactive rays and converts the detected radioactive rays into an electrical signal related to a radiographic image.

### Description of the Related Art

Examples of a commonly used method of radiographic imaging by radiographing apparatuses include a film/screen method and a computed radiography (CR) method. In these methods, a photosensitive film or a phosphor plate that accumulates an image as a latent image is contained in a storage case that is referred to as a film imaging apparatus and standardized by Japanese Industrial Standards (JIS) Z 4905 (ISO 4090), and is used for the radiographic imaging. On the other hand, the use of a radiographing apparatus employing a flat panel detector (FPD) that is a thin film semiconductor material formed on an insulating substrate has been wide spread, and, in a medical image diagnosis, a digital radiographing apparatus is used for capturing a still image and a moving image such as a radioscopic imaging.

In general, the radiographing apparatus is installed and used in a radiation room. However, along with the recent progress of the mounting technology, a thin and light weight portable radiographing apparatus is produced to enable quick and wide-range imaging of a region of an object to be inspected. With the apparatus, there is a case where an engineer carries a portable radiographing apparatus, for example, into a patient's bedroom or an operation room in a hospital other than the radiation room, and performs the radiographic imaging.

Currently, more users ask for sterilization processing on the radiographing apparatus from a view point of prevention of the spread of novel coronaviruses. Conventionally, to perform the sterilization processing on the radiographing apparatus, solvent such as alcohol is used to clean the outer surface of the housing of the radiographing apparatus. However, there are risks of a strength degradation along with a reduction in molecular-weight of a member of the radiographing apparatus, an external appearance deterioration, and a malfunction caused by the solvent entering the inner portion of the radiographing apparatus, due to a chemical reaction by the solvent such as alcohol.

For users, there are disadvantages, such as waste generation and burdens on the users, involved in the sterilization processing that are large as the above described cleaning operation occurs for each patient.

On the other hand, there is evidence of a confirmed sterilization effect occurring on viruses, including the novel coronaviruses, through sterilization processing by irradiation with ultraviolet light, and the sterilization processing has an advantage of lower burdens on the users and no waste generation. Taking such a disinfection treatment (sterilization processing) by irradiation with ultraviolet light into consideration, Japanese Patent Application Laid-open No. 2012-123297 discusses a radiographing apparatus including a housing an outer surface of which is applied with a coating material coated by photocatalyst, to enhance the sterilization effect of the ultraviolet light irradiation.

### SUMMARY OF THE DISCLOSURE

According to an aspect of the present disclosure, a radiographing apparatus includes a radiation detector configured to detect an incident radioactive ray and convert the detected radioactive ray into an electrical signal related to a radiation image, and a housing configured to contain the radiation detector, wherein an improvement to the radiographing apparatus is that an ultraviolet light transmitting member having transmittivity for ultraviolet light is provided at least in a part of an outer housing surface of the housing.

Further features of the present disclosure will become apparent from the following description of exemplary embodiments with reference to the attached drawings.

### BRIEF DESCRIPTION OF THE DRAWINGS

FIGS. 1A and 1B are diagrams illustrating an example of an external appearance of a radiographing apparatus according to a first exemplary embodiment.

FIG. 2 is a diagram illustrating an example of an internal configuration in an A-A cross-section of the radiographing apparatus illustrated in FIG. 1A.

FIGS. 3A, 3B, and 3C are diagrams each illustrating an example of a layer structure of an outer housing surface of a housing of the radiographing apparatus illustrated in FIG. 2.

FIG. 4 is a diagram illustrating an example of an external appearance of a radiographing apparatus according to a second exemplary embodiment.

FIGS. 5A and 5B are diagrams illustrating an example of an internal configuration of the radiographing apparatus in a B-B cross-section illustrated in FIG. 4.

FIG. 6 is a diagram illustrating an example of an internal configuration of the radiographing apparatus in the B-B cross-section illustrated in FIG. 4.

### DESCRIPTION OF THE EMBODIMENTS

A housing of a radiographing apparatus has an area that is not exposed structurally. More specifically, examples of the area that is not exposed structurally include an inner portion of a crack in an outer housing surface of the housing generated along with the use, and an inner portion of a slide member such as a switch button provided on the outer housing surface of the housing. When the area that is not exposed structurally (i.e., area that is structurally hidden) is present on the housing of the radiographing apparatus, it is difficult to irradiate the area with uniform ultraviolet light, and thus it is difficult to obtain a sufficient sterilization effect. In general, an ultraviolet light irradiation apparatus used in a medical site is a general-purpose ultraviolet light irradiation apparatus also used for a surgical instrument or the like and is not dedicated to the radiographing apparatus. Thus, in order to irradiate, with the ultraviolet light, the structurally-hidden area of the housing of the radiographing apparatus, making the ultraviolet light irradiation apparatus complicated and/or large so that the ultraviolet light irradiation apparatus is dedicated for the radiographing apparatus imposes a burden on a user.

Thus, the present disclosure is devised in consideration of such issues, and is directed to a method of performing sterilization processing on a structurally-hidden area of a housing of a radiographing apparatus when a general-purpose ultraviolet light irradiation apparatus is used to perform the sterilization processing to irradiate the radiographing apparatus with the ultraviolet light.

Exemplary embodiments of the present disclosure will now be described with reference to the attached drawings.

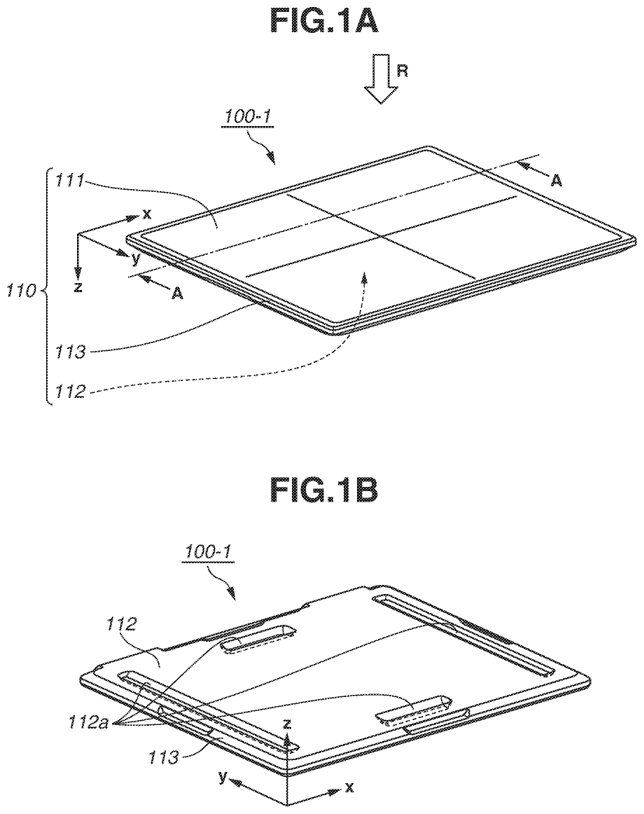

FIGS. 1A and 1B are diagrams illustrating an example of an external appearance of a radiographing apparatus 100-1 according to the first exemplary embodiment. More specifically, FIG. 1A is an example of an external appearance of the radiographing apparatus 100-1 viewed from an incident side of a radioactive ray R that has transmitted through an object to be inspected (not depicted), and FIG. 1B is an example of an external appearance of the radiographing apparatus 100-1 viewed from a rear surface side opposite to the incident side of the radioactive ray R. Further, FIGS. 1A and 1B each illustrate an xyz coordinate system in which an incident direction of the radioactive ray R is defined as a z direction, and two directions orthogonal to the z direction and orthogonal to each other are respectively defined as an x direction and a y direction.

The radiographing apparatus 100-1 includes a housing 110 serving as an outer housing. In the example illustrated in FIG. 1A, the housing 110 of the radiographing apparatus 100-1 includes a front cover 111, a rear cover 112, and a frame 113. In addition, in FIG. 1B, components similar to those illustrated in FIG. 1A are assigned the same symbols.

In the housing 110 of the radiographing apparatus 100-1, a radiation detector and an attachable/detachable battery serving as a power source are provided. The radiation detector detects the incident radioactive ray R and converts the detected radioactive ray R into an electrical signal related to a radiation image formed by the radioactive ray R. The frame 113 located on a side surface of the housing 110 of the radiographing apparatus 100-1 has large openings respectively on a radiation incident surface side of the housing 110 and on a rear surface side opposite to the radiation incident surface side. The front cover 111 is attached to the opening on the radiation incident surface side of the frame 113. The rear cover 112 is attached to the opening on the rear surface side of the frame 113. Since the housing 110 forms the outer housing of the radiographing apparatus 100-1, the components inside the radiographing apparatus 100-1 can be protected. Further, as illustrated in FIG. 1B, a plurality of depressed portions 112a is provided in the rear cover 112 to increase the ease of gripping the radiographing apparatus 100-1 in consideration of the need for portability of the radiographing apparatus 100-1 when a user such as an engineer carries the radiographing apparatus 100-1. As illustrated in FIG. 1B, these depressed portions 112a are desirably provided near the respective corners of the frame 113 and deeper and over a wide area in consideration of the arrangement of other components in the radiographing apparatus 100-1.

For the front cover 111, a carbon fiber reinforced resin is often used because the carbon fiber reinforced resin is a material that easily transmits the radioactive ray R and has a light weight and a high stiffness. For the rear cover 112, a metal material having a light weight and a high stiffness such as an aluminum alloy or a magnesium alloy, or a resin material such as the carbon fiber reinforced resin is desirably used. For the frame 113, a metal material having a light weight and a high stiffness such as an aluminum alloy or a magnesium alloy, or a resin material such as the carbon fiber reinforced resin is used.

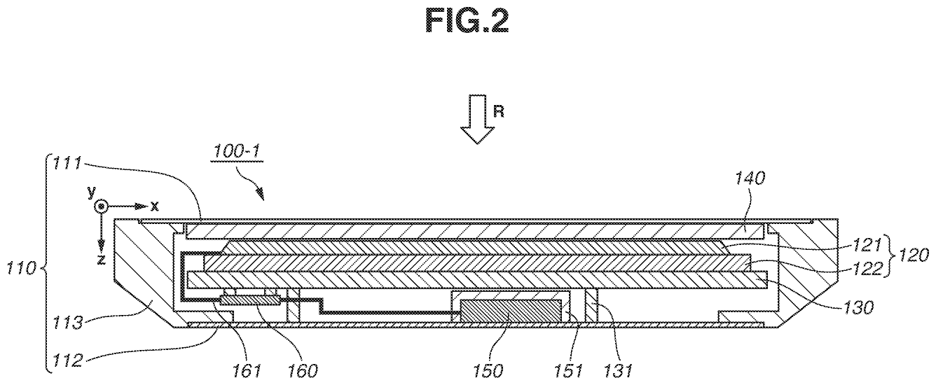

FIG. 2 is a diagram illustrating an example of an internal configuration in an A-A cross-section of the radiographing apparatus 100-1 illustrated in FIG. 1A. In FIG. 2, components similar to those in FIG. 1A are assigned the same symbols, and detailed descriptions thereof are omitted. Further, in FIG. 2, an xyz coordinate system corresponding to the xyz coordinate system illustrated in FIG. 1A is illustrated.

As illustrated in FIG. 2, the housing 110 of the radiographing apparatus 100-1 contains a radiation detector 120, a base 130, a spacer layer 140, a battery 150, a battery holder 151, an electric component 160, and a flexible cable 161.

The radiation detector 120 is a radiation detector that detects the incident radioactive ray R and converts the detected radioactive ray R into a signal related to a radiation image formed by the radioactive ray R. As illustrated in FIG. 2, the radiation detector 120 includes a scintillator layer (phosphor layer) 121 and a sensor 122. The scintillator layer 121 is a layer that converts the incident radioactive ray R into visible light. The sensor 122 is a sensor that converts the visible light generated in the scintillator layer 121 into an electrical signal related to a radiation image. The radiation detector 120 is attached to the radiation incident surface side of the base 130 with an adhesive layer (not illustrated) such as a double-sided adhesive tape therebetween, and arranged together with the base 130 in an integrated manner. In addition, for the adhesive layer (not illustrated), another bonding method such as bonding by an adhesive material may be used instead of the double-sided adhesive tape. In general, the scintillator layer 121 is made of a phosphor material such as Cesium Iodide (CsI) or gadolinium oxysulfide (Gd₂O₂S (GOS)). The sensor 122 employs a flat panel detector, and includes a substrate and photoelectric conversion elements arranged in a two-dimensional manner on a surface of the substrate. The photoelectric conversion elements of the sensor 122 detect and convert the visible light generated by the scintillator layer 121 into an electrical signal. In this way, the radiographing apparatus 100-1 can obtain radiation image data.

The base 130 is a base supporting the radiation detector 120 from the rear surface side opposite to the radiation incident surface side. The base 130 is desirably formed of a material having a light weight and a high stiffness such as a metal (e.g., aluminum alloy or magnesium alloy), or a resin (e.g., the carbon fiber reinforced resin). The base 130 employs a following configuration in each of the z direction that is the incident direction of the radioactive ray R, and the x direction and the y direction that are directions along a plane orthogonal to the z direction to perform a position regulation inside the housing 110 in a state where the base 130 is integrally configured with the radiation detector 120 and other components. First, in the z direction that is the incident direction of the radioactive ray R, the spacer layer 140 is arranged on the radiation incident surface side of the base 130, a rib 131 of the base 130 and a spacer are arranged on the rear surface side of the base 130, and the position regulation can be performed by bringing them into contact with the inner side of the housing 110. In the x direction and the y direction that are the directions along a plane orthogonal to the z direction, the position regulation can be performed by the base 130 and the frame 113 fitted together. Further, in the x direction and the y direction, an impact transmitted to the sensor 122 or the base 130 can be reduced by a buffer material (not illustrated) such as an elastomer material or a foam material arranged in a gap and deformed against an impact applied from a side surface of the housing 110.

The battery 150 is detachably held in the battery holder 151. The battery holder 151 is attached to the rear cover 112, and the battery 150 is attachable and detachable from the rear surface side of the housing 110. Further, an opening may be provided in the rear cover 112, and the battery holder 151 may be attached to the base 130 through the opening. When the battery 150 is mounted on the battery holder 151, power of the battery 150 is supplied to the components of the radiographing apparatus 100-1, such as the sensor 122 and the electric component 160, and the components operate by the power supplied from the battery 150.

The battery 150 is a secondary battery (a battery that is rechargeable and can be used repeatedly), and a capacitor may be used instead.

The electric component 160 is attached to a surface of the base 130 opposite to a surface bonded to the sensor 122, and reads, via the flexible cable 161, and processes the electrical signal converted by the sensor 122 to generate radiation image data. The radiation image data generated by the electric component 160 is transmitted to and displayed on an external display system that has established communication with the radiographing apparatus 100-1. At this time, as a communication method, a wired connection or a wireless connection can be used, and in a case where the wireless communication is used, a 2.4-GHz band or a 5-GHz band is mainly used. With these communication methods, the radiation image data generated by the electric component 160 can be transferred to a personal computer (PC) or a tablet PC, so that the radiation image data can be confirmed.

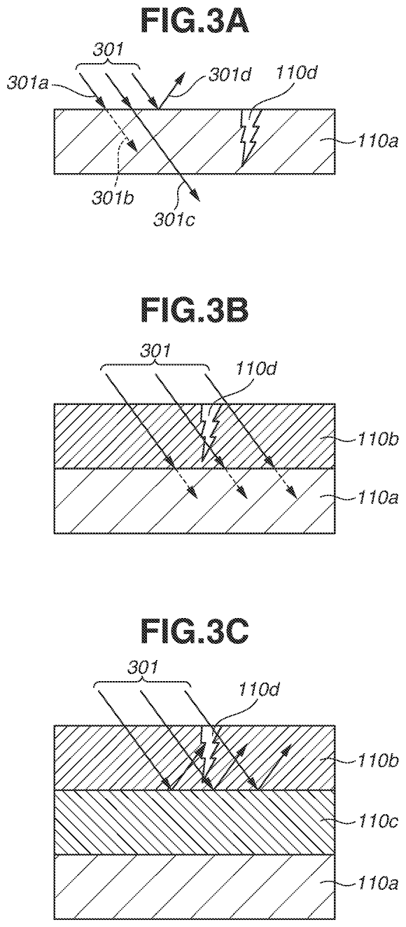

Next, the outer housing surface of the housing 110 of the radiographing apparatus 100-1 illustrated in FIG. 2 will be described. FIGS. 3A, 3B, and 3C are diagrams each illustrating an example of a layer structure of the outer housing surface of the housing 110 of the radiographing apparatus 100-1 illustrated in FIG. 2.

Here, before a description of the layer structure of the outer housing surface of the housing 110 according to the first exemplary embodiment, there is provided a description of a layer structure in which a ultraviolet light transmitting layer with transmissivity for an ultraviolet ray radiated to perform the sterilization processing is not formed with reference to FIG. 3A, as a reference example. FIG. 3A illustrates a surface layer 110a constituting the outer housing surface of the housing 110 including the front cover 111, the rear cover 112, and the frame 113. When ultraviolet light 301 is incident on the surface layer 110a to perform sterilization processing, incident rays 301a of the ultraviolet light 301 are divided into an absorbed ray 301b absorbed within the surface layer 110a, a transmission ray 301c transmitting through the surface layer 110a, and a reflection ray 301d reflected on the surface layer 110a. In this case, assuming that light energies of the incident rays 301a, the absorbed ray 301b, the transmission ray 301c, and the reflection ray 301d of the ultraviolet light 301 are respectively defined as I, A, T, and R, I=A+T+R holds based on the energy conservation law. In a case where the ultraviolet light 301 does not transmit through the surface layer 110a (T≈0), the incident rays 301a of the ultraviolet light 301 become the absorbed ray 301b or the reflection ray 301d.

Next, a description will be provided of a behavior of the ultraviolet light 301 when the ultraviolet light 301 is incident in a case where a crack 110d is present in the outer housing surface of the surface layer 110a. The crack 110d may be caused by an impact or a bending given when the apparatus is used, or may be a cavity generated when the layer is formed. An inner portion of the crack 110d corresponds to the structurally-hidden area (i.e., structurally unexposed area) of the housing 110 of the radiographing apparatus 100-1. Even though the radiographing apparatus 100-1 is irradiated with the ultraviolet light 301 when the above-described crack 110d is present, since only a portion of the ultraviolet light 301 is able to be transmitted through surface layer 110a (transmission ray 301c) such an amount of the transmission ray 301c is not sufficient to provide a sterilizing effect, such that the inner portion of the crack 110d is not irradiated sufficiently with the ultraviolet light 301 while an exposed area of the outer housing surface of the surface layer 110a is irradiated sufficiently with the ultraviolet light 301. As a result, sterilization of the inner portion of the crack 110d can be insufficient.

Next, with reference to FIGS. 3B and 3C, the layer structure of the outer housing surface of the housing 110 according to the first exemplary embodiment will be described. In FIGS. 3B and 3C, components similar to those in FIG. 3A are assigned the same symbols, and detailed descriptions thereof are omitted.

In the layer structure of the outer housing surface of the housing 110 according to the first exemplary embodiment illustrated in FIG. 3B, an ultraviolet light transmitting layer 110b is formed on the outer side of the surface layer 110a. The ultraviolet light transmitting layer 110b corresponds to an ultraviolet light transmitting member having transmittivity to the ultraviolet light 301 radiated to perform the sterilization processing. In other words, the ultraviolet light transmitting layer 110b is formed on the outer housing surface of the housing 110 according to the first exemplary embodiment illustrated in FIG. 3B. The layer structure illustrated in FIG. 3B is different from the layer structure illustrated in FIG. 3A in that, since the layer structure includes the ultraviolet light transmitting layer 110b on the outermost layer, the ultraviolet light 301 transmits through the ultraviolet light transmitting layer 110b, and the inner portion of the crack 110d formed in the outer housing surface is irradiated with the ultraviolet light 301. As a result, a sterilization effect of the same degree as that for the outer most layer can be expected on the inner portion of the crack 110d.

Next, an optical property of the ultraviolet light transmitting layer 110b will be described.

The ultraviolet light transmitting layer 110b can transmit the ultraviolet light 301 radiated for the purpose of the sterilization processing, as a characteristic. More specifically, the ultraviolet light with a wavelength of 200 nm to 300 nm (UV-C) is extremely effective for the sterilization of microbes such as bacteria, viruses, and molds, and it is said that the ultraviolet light can kill or inactivate the microbes by destroying the deoxyribonucleic acids (DNAs) thereof. It is known that the sterilization effect of the ultraviolet light is maximum at a wavelength of about 260 nm. As the ultraviolet light irradiation apparatus for radiating the ultraviolet light 301, a general-purpose ultraviolet light irradiation apparatus having a specification capable of outputting the ultraviolet light having the above-described wavelength is used at medical sites and other facilities. Accordingly, the ultraviolet light transmitting layer 110b is desirably formed of a material with a thickness through which the ultraviolet light 301 with a wavelength lower than the wavelength of 300 nm can transmit. The transmittance of the ultraviolet light 301 in a substance is expressed as T/I using an energy I of the incident rays 301a and an energy T of the transmission ray 301c. In this case, the ultraviolet light transmitting layer 110b according to the present exemplary embodiment desirably has a transmittance of 10% or more for the ultraviolet light 301 having the wavelength of 300 nm.

Specifically, the ultraviolet light transmitting layer 110b having an optical property of the above-described transmittance is desirably formed of the following materials. For example, the ultraviolet light transmitting layer 110b is desirably formed of acrylic resin (polymethylmethacrylate (PMMA)) or polymethyl pentene (polymethylpentene (PMP)) resin with a thickness of several tens of micrometers (μm) to 1 millimeter (mm) order (more specifically, 10 μm to 1 mm). Further, the ultraviolet light transmitting layer 110b may be formed of an organic material such as thin film polycarbonate (PC) resin, or an inorganic material such as glass and crystal, with a thickness of several tens μm to 100 μm order (more specifically, 10 μm to 100 μm). Further, the ultraviolet light transmitting layer 110b may be formed of a material improved by adding an additive agent. For example, in a case where the ultraviolet light transmitting layer 110b is formed of the polymethyl pentene resin, there is an issue of the weather-resistant property, and since the ultraviolet light transmitting layer 110b may be deteriorated due to the irradiation with the ultraviolet light 301, the ultraviolet light transmitting layer 110b is desirably formed of a resin including an additive agent to improve the resistance to the ultraviolet light.

Further, the surface layer 110a is not limited to the material of the outer housing, and may be an antioxidant layer or a film layer formed on the ultraviolet light transmitting layer 110b.

With the configuration of the housing 110 including the ultraviolet light transmitting layer 110b, the improvement of the sterilization effect can be expected, but to obtain the sterilization effect efficiently by the irradiation with the ultraviolet light 301, an ultraviolet light reflection layer that reflects the ultraviolet light 301 may be provided between the surface layer 110a and the ultraviolet light transmitting layer 110b. This configuration will be described with reference to FIG. 3C.

The layer structure of the outer housing surface of the housing 110 according to the first exemplary embodiment illustrated in FIG. 3C is provided with a ultraviolet light reflection layer 110c that reflects the ultraviolet light 301 between the ultraviolet light transmitting layer 110b and the surface layer 110a (or the radiation detector 120 contained inside the housing 110). In the case of the layer structure illustrated in FIG. 3C, the ultraviolet light 301 radiated to perform the sterilization processing transmits through the ultraviolet light transmitting layer 110b, and the inner portion of the crack 110d formed in the outer housing surface of the housing 110 is irradiated with the transmitted ultraviolet light 301, in a similar manner to that in FIG. 3B. In addition, in the case of the layer structure illustrated in FIG. 3C, the ultraviolet light 301 that has transmitted through the ultraviolet light transmitting layer 110b is reflected by the ultraviolet light reflection layer 110c, and enters the ultraviolet light transmitting layer 110b again. In this way, sterilization by the ultraviolet light 301 that has transmitted through the ultraviolet light transmitting layer 110b and reflected by the ultraviolet light reflection layer 110c can be expected in addition to the sterilization by the ultraviolet light 301 directly radiated from the ultraviolet light irradiation apparatus. The reflectance of the ultraviolet light reflection layer 110c according to the present exemplary embodiment is desirably 80% or more for the ultraviolet light 301 having the wavelength of 300 nm. More specifically, the ultraviolet light reflection layer 110c according to the present exemplary embodiment is desirably formed of a metal material such as an aluminum alloy.

The radiographing apparatus 100-1 according to the first exemplary embodiment described above is provided with the ultraviolet light transmitting layer 110b corresponding to an ultraviolet light transmitting member having transmittivity for the ultraviolet light 301 in at least a part of the exposed area of the housing 110.

With this configuration, the inner portion of the crack 110d generated in the outer housing surface of the housing 110 corresponding to the structurally-hidden area of the housing 110 of the radiographing apparatus 100-1 can be irradiated with the ultraviolet light 301 through the ultraviolet light transmitting layer 110b. In this way, the sterilization processing using the ultraviolet light 301 can be performed on the inner portion of the crack 110d corresponding to the structurally-hidden area of the housing 110 of the radiographing apparatus 100-1. Thus, in a similar manner to the outer housing surface of the housing 110, the inner portion of the crack 110d can obtain the sterilization effect using the ultraviolet light 301. Further, as the ultraviolet light irradiation apparatus for radiating the ultraviolet light 301, a general-purpose ultraviolet light irradiation apparatus not dedicated to the radiographing apparatus 100-1 can be used, and thus it is possible to prevent the ultraviolet light irradiation apparatus from becoming complicated and/or large.

Next, a second exemplary embodiment according to the present disclosure will be described. In the second exemplary embodiment described below, items identical with those in the first exemplary embodiment are not described, and items different from those in the first exemplary embodiment will be described.

In the first exemplary embodiment described above, the description is provided of the case where the inner portion of the crack 110d generated in the outer housing surface of the housing 110 is used as the example of the structurally-hidden area of the housing 110 of the radiographing apparatus 100-1. On the other hand, in the second exemplary embodiment, as an example of a structurally-hidden area of the housing 110 of a radiographing apparatus, an inner portion of a slide member such as a switch button provided on the outer housing surface of the housing 110 is used. The sterilization effect can also be obtained in the inner portion of the slide member by the irradiation with the ultraviolet light 301.

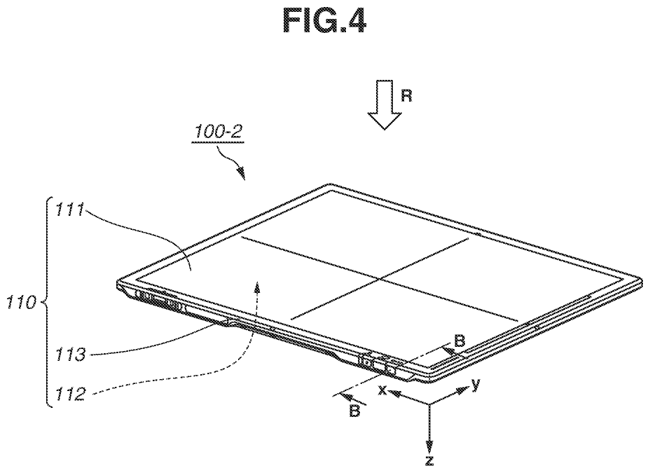

FIG. 4 is a diagram illustrating an example of an external appearance of a radiographing apparatus 100-2 according to the second exemplary embodiment. More specifically, FIG. 4 is an example of an external appearance of the radiographing apparatus 100-2 viewed from a direction in which the radioactive ray R that has transmitted through an object to be inspected enters. In FIG. 4, components similar to those illustrated in FIGS. 1A and 1B are assigned the same symbols, and detailed descriptions thereof are omitted. Further, in FIG. 4, an xyz coordinate system corresponding to the xyz coordinate system illustrated in FIG. 1A is illustrated.

As illustrated in FIG. 4, the radiographing apparatus 100-2 is provided with a switch button 210 corresponding to the slide member operated by a user in an exposed area of the housing 110 (more specifically, the frame 113 constituting a side surface of the housing 110). For example, the user presses the switch button 210 to switch the power status of the radiographing apparatus 100-2, or to shift the mode of the radiographing apparatus 100-2 from a low-power mode to a standby mode ready to capture an image.

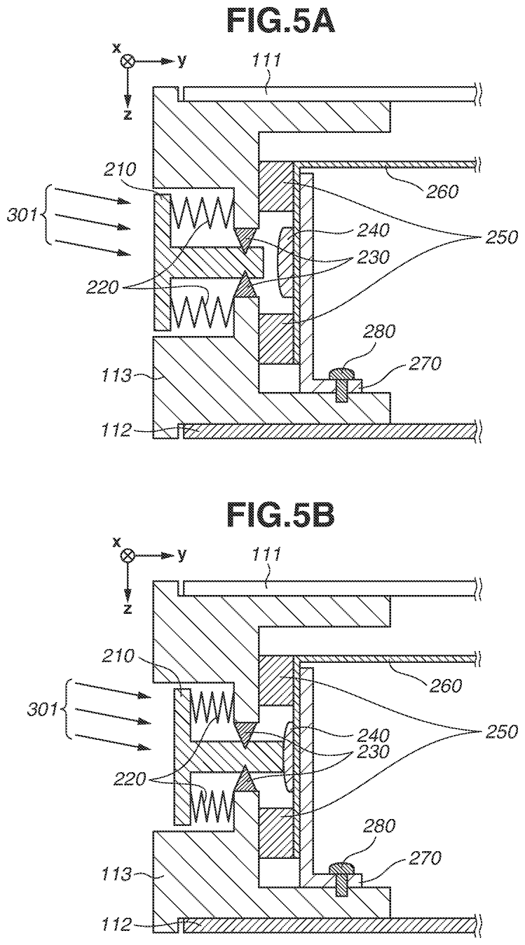

FIGS. 5A and 5B are diagrams illustrating an example of an internal configuration of the radiographing apparatus 100-2 in a B-B cross-section illustrated in FIG. 4. In FIGS. 5A and 5B, components similar to those in FIGS. 2 to 4 are assigned the same symbols, and detailed descriptions thereof are omitted. Further, in each of FIGS. 5A and 5B, an xyz coordinate system corresponding to the xyz coordinate system in FIG. 4 is illustrated.

As illustrated in FIG. 5A, the radiographing apparatus 100-2 includes the switch button 210, a switch button spring 220, a sealing member 230, a tact switch 240, an ultraviolet light diffusion prevention component 250, a flexible printed circuit board (FPC) 260, an FPC fixing member 270, and a screw 280.

A depressed portion is provided in a part of the frame 113. The switch button 210 is fixed to the frame 113 via the switch button spring 220 having a spring function, to be contained in the depressed portion provided in the frame 113.

In the inner portion of the frame 113 (inner portion of the switch button 210), the FPC 260 on which the tact switch 240 is mounted is fixed by the FPC fixing member 270, and the FPC fixing member 270 is fastened to the frame 113 with the screw 280. When the user presses the switch button 210, as illustrated in FIG. 5B, the switch button spring 220 deforms in a compression direction, the switch button 210 slides in a direction toward the inside of the housing 110, and a tip of the switch button 210 presses the tact switch 240. A connector terminal of the FPC 260 extends toward the inner portion of the frame 113 and is connected to an electric substrate or the like (not illustrated) contained in the housing 110 of the radiographing apparatus 100-2. When the tact switch 240 is pressed, the switching processing described above is performed on the connected electric substrate.

A through-hole is provided at a position facing the tip of the switch button 210 contained in the depressed portion of the frame 113. The sealing member 230 is provided in the radiographing apparatus 100-2 to prevent water from entering the inner portion of the housing 110 through the through-hole. The sealing member 230 has a valve shape, and the sealing property is secured by the valve shape being crushed between the frame 113 and the switch button 210. The switch button spring 220 may be fixed to the bottom surface of the frame 113 via a bonding member such as a double-sided adhesive tape and an adhesive agent, or the bottom surface of the frame 113 may have a depression-protrusion shape that fits to the switch button spring 220.

As described above, the switch button 210 is a slide member that slides by a pressing operation of a user. Accordingly, a clearance needs to be provided between the outer periphery of the switch button 210 and the depressed portion of the frame 113. On the other hand, dusts, body fluids of a patient, and solvent used in cleaning may enter the inner portion (between the frame 113 and the switch button 210) of the switch button 210 through the clearance, and there is a risk that the dusts, the body fluids of a patient, and the solvent may remain or accumulate in the inner portion of the switch button 210. For this reason, the sterilization processing by the irradiation with the ultraviolet light 301 needs to be performed on the area of the inner portion (between the frame 113 and the switch button 210) of the switch button 210. If the switch button 210 is formed of a member through which the ultraviolet light 301 does not transmit, since the radiated ultraviolet light 301 is absorbed or reflected by the switch button 210, a region in the inner portion of the switch button 210 is not irradiated with the ultraviolet light 301, and thus the sterilization cannot be performed.

In view of the foregoing, in the radiographing apparatus 100-2 according to the second exemplary embodiment, a switch button 211, which is a slide member provided in the exposed area of the housing 110, is formed of an ultraviolet light transmitting member having transmittivity to the ultraviolet light 301. In this case, the transmittance of the switch button 211, which is the ultraviolet light transmitting member, is desirably 10% or more for the ultraviolet light 301 with the wavelength of 300 nm, similar to the ultraviolet light transmitting layer 110b described above in the first exemplary embodiment. In this case, the switch button 211 is desirably formed of a material such as acrylic resin (PMMA) or polymethyl pentene (PMP) resin from a viewpoint of the transmittance for the ultraviolet light 301, similar to the ultraviolet light transmitting layer 110b according to the first exemplary embodiment described above.

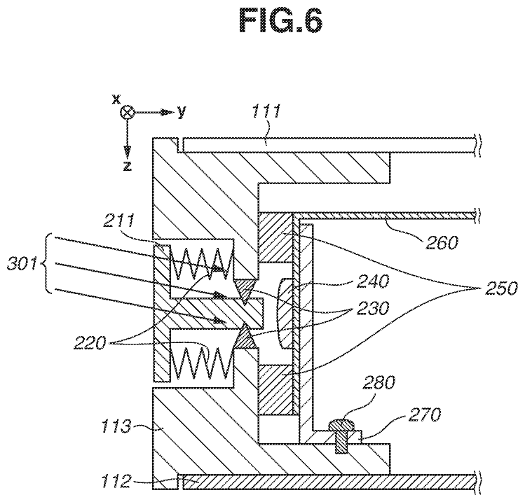

FIG. 6 is a diagram illustrating an example of an internal configuration of the radiographing apparatus 100-2 in the B-B cross-section illustrated in FIG. 4. More specifically, FIG. 6 is a diagram illustrating an example of a behavior of the radiographing apparatus 100-2 according to the second exemplary embodiment at a time of being irradiated with the ultraviolet light 301. In FIG. 6, components similar to those in FIGS. 2 to 5B are assigned the same symbols, and detailed descriptions thereof are omitted. Further, in FIG. 6, an xyz coordinate system corresponding to the xyz coordinate system illustrated in each of FIGS. 4, 5A, and 5B is illustrated.

As illustrated in FIG. 6, when the radiographing apparatus 100-2 is irradiated with the ultraviolet light 301, the ultraviolet light 301 transmits through the switch button 211 and enters the inner portion (between the frame 113 and the switch button 211) of the switch button 211. Thus, in the radiographing apparatus 100-2 illustrated in FIG. 6, the sterilization processing can be performed on the region in the inner portion (between the frame 113 and the switch button 211) of the switch button 211 by irradiation with the ultraviolet light 301.

On the other hand, there is a possibility that the ultraviolet light 301 that has transmitted through the central portion of the switch button 211 enters the inner portion of the housing 110 through the above-described through-hole in the frame 113, and diffuse. Thus, the radiographing apparatus 100-2 according to the present exemplary embodiment is provided with the ultraviolet light diffusion prevention component 250 between the switch button 211 that is an ultraviolet light transmitting member and the FPC 260 (or the radiation detector 120 contained in the inner portion of the housing 110). The ultraviolet light diffusion prevention component 250 has a function of preventing the ultraviolet light 301 that has transmitted through the switch button 211, which is an ultraviolet light transmitting member, from diffusing in the inner portion of the housing 110. The ultraviolet light diffusion prevention component 250 is formed of, for example, a urethane based foam material to secure the ultraviolet light diffusion prevention function by being crushed between the internal surface of the frame 113 and the FPC fixing member 270.

The radiographing apparatus 100-2 according to the second exemplary embodiment described above is provided with the switch button 211 as the slide member corresponding to the ultraviolet light transmitting member having transmittivity for the ultraviolet light 301 in at least a part of the exposed area of the housing 110.

With this configuration, the inner portion of the switch button 211 (slide member) corresponding to the structurally-hidden area of the housing 110 of the radiographing apparatus 100-2 can be irradiated with the ultraviolet light 301 through the switch button 211. In this way, the sterilization processing using the ultraviolet light 301 can be performed on the inner portion of the switch button 211 (slide member) corresponding to the structurally-hidden area of the housing 110 of the radiographing apparatus 100-2. Thus, in a similar manner to the outer housing surface of the housing 110, the inner portion of the switch button 211 (slide member) can obtain the sterilization effect using the ultraviolet light 301. Further, as the ultraviolet light irradiation apparatus for radiating the ultraviolet light 301, a general-purpose ultraviolet light irradiation apparatus not dedicated to the radiographing apparatus 100-2 can be used, and thus it is possible to prevent the ultraviolet light irradiation apparatus from becoming complicated and/or large.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2022-062973, filed Apr. 5, 2022, which is hereby incorporated by reference herein in its entirety.

What is claimed is:

1. A radiographing apparatus comprising:
a radiation detector configured to detect an incident radioactive ray and convert the detected radioactive ray into an electrical signal related to a radiation image; and
a housing configured to contain the radiation detector,
wherein an improvement to the radiographing apparatus is that an ultraviolet light transmitting member having transmittivity for ultraviolet light is provided at least in a part of an outer housing surface of the housing.

2. The radiographing apparatus according to claim 1, wherein a transmittance of the ultraviolet light transmitting member is 10% or more for the ultraviolet light with a wavelength of 300 nm.

3. The radiographing apparatus according to claim 1, wherein a thickness of the ultraviolet light transmitting member is 10 μm to 1 mm.

4. The radiographing apparatus according to claim 1, wherein the ultraviolet light transmitting member is formed of acrylic resin, polymethyl pentene resin, polycarbonate resin, glass, or crystal.

5. The radiographing apparatus according to claim 1, wherein the ultraviolet light transmitting member is formed on the outer housing surface of the housing as an ultraviolet light transmitting layer.

6. The radiographing apparatus according to claim 5, wherein an ultraviolet light reflection layer is provided between the ultraviolet light transmitting layer and the radiation detector.

7. The radiographing apparatus according to claim 6, wherein a reflectance of the ultraviolet light reflection layer is 80% or more for the ultraviolet light with a wavelength of 300 nm.

8. The radiographing apparatus according to claim 1, wherein the ultraviolet light transmitting member is a slide member provided on the outer housing surface of the housing.

9. The radiographing apparatus according to claim 8, wherein the slide member is provided in a depressed portion provided in a part of the outer housing surface of the housing via a member with a spring function.

10. The radiographing apparatus according to claim 1, further comprising an ultraviolet light diffusion prevention component between the ultraviolet light transmitting member and the radiation detector, the ultraviolet light diffusion prevention component being configured to prevent the ultraviolet light having transmitted through the ultraviolet light transmitting member from diffusing in an inner portion of the housing.

11. The radiographing apparatus according to claim 1, wherein the ultraviolet light transmitting member is made of resin containing an additive agent for improving resistance to the ultraviolet light.

12. A radiographing apparatus comprising:
a radiation detector configured to detect an incident radioactive ray and convert the detected radioactive ray into an electrical signal related to a radiation image; and
a housing configured to contain the radiation detector,
wherein at least a part of an outer surface of the housing comprises an ultraviolet light transmitting member having transmittivity for ultraviolet light.
